(19) United States
(12) Patent Application Publication
(10) Pub. No.: US 2021/0283244 A1
(43) Pub. Date: Sep. 16, 2021

(54) ADENOVIRUS-VECTORED VACCINE FOR PREVENTING SARS-COV-2 INFECTION

(71) Applicant: GUANGZHOU N BIOMED LTD., Guangzhou, Guangdong (CN)

(72) Inventors: Ling Chen, Guangzhou, Guangdong (CN); Suhua Guan, Guangzhou, Guangdong (CN); Chenchen Yang, Guangzhou, Guangdong (CN); Xiaolin Liu, Guangzhou, Guangdong (CN)

(21) Appl. No.: 17/260,820

(22) PCT Filed: Aug. 19, 2020
(86) PCT No.: PCT/CN2020/110085

§ 371 Date: Jan. 15, 2021

(30) Foreign Application Priority Data
Feb. 23, 2020 (CN) 202010110070.8
Mar. 5, 2020 (CN) 202010145657.2

(51) Int. Cl. A61K 39/215; C12N 15/86; A61P 31/14

(52) U.S. Cl. CPC A61K 39/215; A61K 2039/53; A61P 31/14; C12N 15/86

(57) ABSTRACT

The present disclosure discloses an adenovirus vectored vaccine for preventing SARS-CoV-2 infection, which comprises a nucleotide sequence as shown by SEQ ID NO: 1. According to some embodiments of the present disclosure, the vaccine comprises an S protein-coding nucleotide sequence which is easily expressed in human cells, and can produce more S proteins. The vaccine of the present disclosure is expected to be used as a recombinant viral vaccine for preventing SARS-CoV-2 infection. Some embodiments of the present disclosure have better safety.

### TECHNICAL FIELD

The present disclosure relates to an adenovirus-vectored vaccine for preventing SARS-CoV-2 infection.

### BACKGROUND

Novel Severe Acute Respiratory Syndrome Coronavirus 2 (SARS-CoV-2) has a latent period up to 24 days, and is highly infectious. Unlike the virus that caused Severe Acute Respiratory Syndrome (SARS), some cases infected with SARS-CoV-2 have infectivity during the latent period, while some SARS-CoV-2 carriers do not show any obvious symptoms. This increases the difficulty of epidemic prevention and control. As of 20 Jul. 2020, there have been close to 15 million confirmed cases, including deaths of above 600,000, all over the world. So far there is no specific medicine available for COVID-19. To control the epidemic, most countries mandated quarantine and closed unnecessary facilities, which resulted in huge economic loss. Therefore, it has become the most urgent and major need to rapidly develop preventive vaccines that can improve herd-immunity level and block the transmission of the virus.

Currently, there is no specific antiviral medicine and preventive vaccine against this novel coronavirus, in China and other countries. Preventing and blocking the viral transmission are the keys to control the epidemic. It is known that vaccines have played an irreplaceable role in eliminating various infectious diseases. The published results obtained from the genome alignment of a dozen of SARS-CoV-2 viruses showed that the difference among the viruses is very small, and no mutation has been found. Therefore, the outbreak of new epidemic would be greatly contained, if SARS-CoV-2 vaccines are successfully developed.

Adenovirus is a commonly used vector in vaccine development and gene therapy, and is widely applied in biomedical field. The adenovirus has lower toxicity as compared with other vectors, such as bacteria- or other viruses-based vectors, and only causes mild flu-like symptoms. Ad5-empty vectors are replication-defective Ad5 vectors lacking the E1 and E3 genes, and carry no foreign gene. Gene therapy products using Ad5 as carriers have been proved to be safe in many clinical trials.

SARS-CoV-2 has a complete genome as shown by NC 045512.2, in which the sequence of nucleic acids 21563 to 25384 is a coding sequence for Spike protein (S). The S protein is a major structural protein of a virion, and plays an important role in mediating the binding of the virion to a host cell receptor and inducing neutralizing antibodies. Vaccines, which use the S protein as an antigen, comprise nucleic acid-, subunit- and virus-vectored vaccines, and the efficacy of such vaccines are determined by the expression level and protein structure of the S protein.

However, it was shown by experiments that the gene coding for the Spike protein of natural SARS-CoV-2 has low expression level in human body. If a vaccine adopts natural genetic codons to express the S protein as the antigen, this vaccine would not produce sufficient antigen, and thus would be ineffective or have low titer, thereby being unable to fight the virus infection.

### SUMMARY

The present disclosure is intended to overcome the above shortcomings of the prior art, and provide an optimized nucleotide sequence for coding the S protein. As the optimized nucleotide sequence is integrated into 1) a DNA plasmid or 2) an adenovirus vector, the S protein can be produced at a higher level after the plasmid or adenovirus vector is transfected into human cells. Therefore, the optimized nucleotide sequence for coding the S protein is expected to be used as an antigen gene for a nucleic acid vaccine or a recombinant viral vaccine to prevent SARS-CoV-2 infection.

The technical solutions adopted by the present disclosure are as follows.

In a first aspect of the present disclosure, provided is a vaccine for preventing SARS-CoV-2 infection, wherein the vaccine comprises:

a) a nucleotide sequence as shown by SEQ ID NO: 1; or

b) a nucleotide sequence having at least 80%, 85%, 90%, 95% or 100% homology with the nucleotide as shown by SEQ ID NO: 1.

In some embodiments, said nucleotide sequence can express a protein in a human-derived cell or human body.

In some embodiments, in the human body, said protein is capable of:

inducing an immune response;

generating a molecular biology reporter;

generating a trace molecule for detection;

regulating a gene function; or

functioning as a therapeutic molecule.

The induced immune response may include antibody- and cell-mediated immune responses.

In some embodiments, the vaccine may further include a pharmaceutically-acceptable adjuvant, carrier, diluent or excipient.

In some embodiments, the vaccine may further include at least one medicament having therapeutic effect on coronavirus disease 2019 (COVID-19).

In a second aspect of the present disclosure, provided is an expression vector which comprises the nucleotide sequence according to the first aspect of the present disclosure.

In some embodiments, the nucleotide sequence according to the first aspect of the present disclosure may have a transcription direction opposite to those of the other genes in the vector. This can further ensure that the expressed S protein has a higher level of purity.

In some embodiments, the vector may be a DNA plasmid, an RNA expression plasmid, or a viral vector.

In some embodiments, the viral vector may be an adenovirus vector. In particular, the adenovirus vector may be a replication-defective Ad5 vector.

In a third aspect of the present disclosure, provided is use of the vaccine according to the first aspect of the present disclosure, which comprises:

preparing a COVID-19 detection reagent; and

preparing a gene-function regulator.

In a fourth aspect of the present disclosure, provided is a method for preventing or treating COVID-19 infection, which comprises administering a prophylactically or therapeutically effective amount of the vaccine according to the first aspect of the present disclosure to a human.

The beneficial effects of the present disclosure are as follows.

The vaccine according to some embodiments of the present disclosure has an S protein-coding nucleotide sequence, which is easily expressed in human cells and can induce the production of the S protein at a higher level. Such vaccine is expected to be used as a recombinant viral vaccine for preventing SARS-CoV-2 infection.

The vaccine according to some embodiments of the present disclosure has better safety.

As shown by the animal experiments performed in some examples of the present disclosure, the vaccine can produce a high titer of antibodies and cellular immunity after immunizing mice and macaque monkeys (Macaca mulatta). The macaque monkeys were immunized with the vaccine through single intramuscular (I.M.) or intra-nasal/oral (I.N.) administration, and were challenged with the SARS-Cov-2 virus 30 days after the immunization. The results showed that the immunized macaque monkeys were fully resistant to the virus infection. When the macaque monkeys were intramuscularly immunized once with 1/10 dose (1×10¹⁰ vp/animal) of the vaccine and challenged with the SARS-Cov-2 virus 8 weeks after the immunization, the immunized macaque monkeys were also fully resistant to the virus infection. For the vaccinated macaque monkeys, no lung damage caused by antibody-dependent enhancement (ADE) or the virus was observed after receiving the virus challenge. For most of the vaccinated macaque monkeys, the antibody level did not increase after receiving the virus challenge. It indicates that the virus was eliminated quickly, resulting in no stimulation effect on the immune system. Different from the whole-virus inactivated vaccine, the vaccine of the present disclosure, in addition to stimulating humoral immunity, also induces cellular immunity, which can further improve the protective effect on body function. Furthermore, since the intra-nasal/oral immunization also obtained good protective effect, it suggests the feasibility of mucosal- and non-injection immunization.

### BRIEF DESCRIPTION OF THE DRAWINGS

FIG. 1 shows the expression of S protein after transfecting human-derived cells with a plasmid containing the codon-optimized nucleotide sequence of the S gene.

FIG. 2 shows the experimental results of animal immunogenicity evaluation, which illustrates that Ad5-NB2 can induce the production of the antibody in mice.

FIG. 3 shows the detection results of the binding antibody in serum of the macaque monkeys at different times after immunization, in which a: the detection results of the binding antibodies in serum of the macaque monkeys at different times after immunization; and b: the detection results of the binding antibodies in serum of the macaque monkeys 18 days after immunization.

FIG. 4 shows the titer of neutralizing antibodies in serum of the immunized macaque monkeys against live novel coronaviruses.

FIG. 5 shows the ELISpot results of the peripheral blood mononuclear cell (PBMCs) of the macaque monkeys 18 days after immunization.

FIG. 6 shows the copy number of virus genome from the pharyngeal swabs of the macaque monkeys after virus challenge, in which a: the copy number changes of the virus genome in a control group after virus challenge; b: the copy number changes of the virus genome in an intramuscular injecting high-dose group after virus challenge; c: the copy number changes of the virus genome in an intramuscular injecting low-dose group after virus challenge; and d: the copy number changes of the virus genome in an intra-nasal/oral group after virus challenge.

FIG. 7 shows the viral load peaks from the pharyngeal swabs of vaccinated and non-vaccinated macaque monkeys, in which each circle represents the viral load peak of a macaque monkey; each diamond represents a macaque monkey intramuscularly vaccinated with a high dose vaccine; each square represents a macaque monkey vaccinated through intra-nasal/oral administration; each triangle represents a macaque monkey intramuscularly vaccinated with a low dose vaccine; and each black circle represents a non-vaccinated macaque monkey.

FIG. 8 shows the copy number of virus genome in different tissues of macaque monkeys after virus challenge.

FIG. 9 shows the pathological results of the lung of the macaque monkeys after virus challenge, in which a: the pathological sections of the lung of the macaque monkeys from the control group after virus challenge (HE staining); b: the pathological sections of the lung of the macaque monkeys from the control group without virus challenge (HE staining); c: the pathological sections of the lung of the macaque monkeys from the high-dose intramuscularly administrated group (1×10¹¹ VP) after virus challenge (HE staining); d: the pathological sections of the lung of the macaque monkeys from the intra-nasally/orally immunized group (5×10¹⁰ vp/route) after virus challenge (HE staining); and e: the pathological sections of the lung of the macaque monkeys from the low-dose intramuscularly administrated group (1×10¹⁰ vp) after virus challenge (HE staining).

FIG. 10 shows the changes of the anti-SARS-CoV-2 neutralizing antibodies of the macaque monkeys before and after virus challenge, in which a: the changes of the anti-SARS-CoV-2 neutralizing antibodies of the intramuscular administration high-dose group before and after virus challenge; b: the changes of the anti-SARS-CoV-2 neutralizing antibodies of the intra-nasal/oral administration group before and after virus challenge; c: the changes of the anti-SARS-CoV-2 neutralizing antibodies of the intramuscular administration low-dose group before and after virus challenge; and d: the changes of the anti-SARS-CoV-2 neutralizing antibodies of the control group before and after virus challenge.

FIG. 11 shows the changes of the anti-Ad5 neutralizing antibodies of the macaque monkeys before and after immunization, in which a: the changes of the anti-Ad5 neutralizing antibodies of the intramuscular administration high-dose group before and after immunization; b: the changes of the anti-Ad5 neutralizing antibodies of the intramuscular administration low-dose group before and after immunization; c: the changes of the anti-Ad5 neutralizing antibodies of the intra-nasal/oral administration group before and after immunization; and d: the changes of the anti-Ad5 neutralizing antibodies of the control group before and after immunization.

### DETAILED DESCRIPTION

The Spike protein (S) of SARS-CoV-2 has an amino acid sequence as shown by YP_009724390.1, designated as NB1.

Precursor mRNA in eukaryotic cells can produce various mRNA splice variants through different splicing manners (by selecting different combination of splice sites), eventually leading to different proteins produced from the same gene sequence. This is very unfavorable for the expression of a protein. The inventor performed codon optimization to the wild-type natural nucleotide sequence and removed potential variable cleavage sites based on self-owned technology, thereby ensuring the uniqueness of expressed protein and reducing the difficulty in subsequent protein purification. The optimized nucleotide sequence is designated as NB2, and is as shown by SEQ ID NO: 1:

(SEQ ID NO.: 1)
ATGTTCGTGTTTCTGGTGCTGCTGCCTCTGGTGAGCTCCCAGTGCGTGA
ACCTGACCACAAGGACCCAGCTGCCACCTGCCTATACCAATAGCTTCAC
ACGGGGCGTGTACTATCCCGACAAGGTGTTTAGATCTAGCGTGCTGCAC
TCCACCCAGGATCTGTTTCTGCCTTTCTTTTCTAACGTGACATGGTTCC
ACGCCATCCACGTGTCCGGCACCAATGGCACAAAGCGGTTCGACAATCC
AGTGCTGCCCTTTAACGATGGCGTGTACTTCGCCTCCACCGAGAAGTCT
AACATCATCAGAGGCTGGATCTTTGGCACCACACTGGACAGCAAGACCC
AGTCCCTGCTGATCGTGAACAATGCCACAAACGTGGTCATCAAGGTGTG
CGAGTTCCAGTTTTGTAATGATCCCTTCCTGGGCGTGTACTATCACAAG
AACAATAAGTCTTGGATGGAGAGCGAGTTTAGGGTGTATTCCTCTGCCA
ACAATTGCACCTTTGAGTACGTGAGCCAGCCTTTCCTGATGGACCTGGA
GGGCAAGCAGGGCAATTTCAAGAACCTGAGGGAGTTCGTGTTTAAGAAT
ATCGATGGCTACTTCAAGATCTACTCCAAGCACACACCAATCAACCTGG
TGCGCGACCTGCCACAGGGCTTCTCTGCCCTGGAGCCACTGGTGGATCT
GCCCATCGGCATCAACATCACCCGGTTTCAGACACTGCTGGCCCTGCAC
AGAAGCTACCTGACCCCAGGCGACAGCTCCTCTGGATGGACAGCAGGAG
CTGCCGCCTACTATGTGGGCTATCTGCAGCCCCGCACCTTCCTGCTGAA
GTACAACGAGAATGGCACCATCACAGACGCAGTGGATTGCGCCCTGGAC
CCCCTGTCTGAGACCAAGTGTACACTGAAGAGCTTTACAGTGGAGAAGG
GCATCTACCAGACCAGCAACTTCAGGGTGCAGCCAACAGAGTCCATCGT
GCGCTTTCCCAATATCACCAACCTGTGCCCTTTTGGCGAGGTGTTCAAT
GCCACACGCTTCGCCAGCGTGTACGCCTGGAATAGGAAGCGCATCTCCA
ACTGCGTGGCCGACTATTCTGTGCTGTACAACAGCGCCTCCTTCTCTAC
CTTTAAGTGTTATGGCGTGAGCCCCACCAAGCTGAATGATCTGTGCTTT
ACAAACGTGTACGCCGATTCCTTCGTGATCAGGGGCGACGAGGTGCGCC
AGATCGCACCAGGACAGACCGGCAAGATCGCAGACTACAATTATAAGCT
GCCTGACGATTTCACAGGCTGCGTGATCGCCTGGAACTCTAACAATCTG
GATAGCAAAGTGGGCGGCAACTACAATTATCTGTACCGGCTGTTTAGAA
AGTCTAATCTGAAGCCATTCGAGCGGGACATCTCCACCGAGATCTACCA
GGCCGGCTCTACACCCTGCAATGGCGTGGAGGGCTTTAACTGTTATTTC
CCTCTGCAGTCCTACGGCTTCCAGCCAACCAACGGCGTGGGCTATCAGC
CCTACAGAGTGGTGGTGCTGTCTTTTGAGCTGCTGCACGCACCTGCAAC
CGTGTGCGGCCCAAAGAAGAGCACAAATCTGGTGAAGAACAAGTGCGTG
AACTTCAACTTCAACGGACTGACCGGCACAGGCGTGCTGACCGAGAGCA
ACAAGAAGTTCCTGCCATTTCAGCAGTTCGGCAGGGACATCGCAGATAC
CACAGACGCCGTGCGCGACCCTCAGACCCTGGAGATCCTGGACATCACA
CCATGTTCCTTCGGCGGCGTGTCTGTGATCACCCCAGGCACCAATACAT
CCAACCAGGTGGCCGTGCTGTATCAGGACGTGAATTGCACAGAGGTGCC
CGTGGCAATCCACGCAGATCAGCTGACCCCTACATGGCGGGTGTACTCT
ACCGGCAGCAACGTGTTCCAGACAAGAGCCGGATGCCTGATCGGAGCAG
AGCACGTGAACAATAGCTATGAGTGCGACATCCCTATCGGCGCCGGCAT
CTGTGCCTCCTACCAGACCCAGACAAACTCCCCAAGGAGAGCCCGGTCT
GTGGCCAGCCAGTCCATCATCGCCTATACCATGAGCCTGGGCGCCGAGA
ACAGCGTGGCCTACTCCAACAATTCTATCGCCATCCCTACCAACTTCAC
AATCAGCGTGACCACAGAGATCCTGCCAGTGAGCATGACCAAGACATCC
GTGGACTGCACCATGTATATCTGTGGCGATTCCACAGAGTGTTCTAACC
TGCTGCTGCAGTACGGCTCCTTTTGCACCCAGCTGAATAGAGCCCTGAC
AGGCATCGCCGTGGAGCAGGACAAGAACACCCAGGAGGTGTTCGCCCAG
GTGAAGCAGATCTACAAGACACCACCCATCAAGGACTTTGGCGGCTTCA
ACTTCAGCCAGATCCTGCCCGATCCTAGCAAGCCATCCAAGCGGTCTTT
TATCGAGGACCTGCTGTTCAACAAGGTGACCCTGGCCGATGCCGGCTTC
ATCAAGCAGTATGGCGATTGTCTGGGCGACATCGCCGCCAGAGACCTGA
TCTGCGCCCAGAAGTTTAATGGCCTGACCGTGCTGCCTCCACTGCTGAC
AGATGAGATGATCGCACAGTACACCTCTGCCCTGCTGGCCGGCACCATC
ACAAGCGGATGGACATTCGGCGCAGGAGCCGCCCTGCAGATCCCCTTTG
CCATGCAGATGGCCTATCGGTTCAACGGCATCGGCGTGACCCAGAATGT
GCTGTACGAGAACCAGAAGCTGATCGCCAATCAGTTTAACAGCGCCATC
GGCAAGATCCAGGACTCTCTGAGCTCCACCGCCAGCGCCCTGGGCAAGC
TGCAGGATGTGGTGAATCAGAACGCCCAGGCCCTGAATACACTGGTGAA
GCAGCTGTCTAGCAACTTCGGCGCCATCTCCTCTGTGCTGAATGACATC
CTGAGCCGGCTGGACAAGGTGGAGGCAGAGGTGCAGATCGACCGGCTGA
TCACCGGCAGACTGCAGTCCCTGCAGACCTACGTGACACAGCAGCTGAT
CAGGGCAGCAGAGATCAGGGCCTCTGCCAATCTGGCCGCCACAAAGATG
AGCGAGTGCGTGCTGGGACAGTCCAAGAGGGTGGACTTTTGCGGCAAGG
GCTATCACCTGATGAGCTTCCCACAGTCCGCCCCTCACGGAGTGGTGTT
TCTGCACGTGACCTACGTGCCAGCCCAGGAGAAGAACTTCACCACAGCC
CCCGCCATCTGTCACGATGGCAAGGCCCACTTTCCTAGGGAGGGCGTGT
TCGTGAGCAACGGCACCCACTGGTTTGTGACACAGCGCAATTTCTACGA
GCCACAGATCATCACCACAGACAATACCTTCGTGTCCGGCAACTGCGAC
GTGGTCATCGGCATCGTGAACAATACAGTGTATGATCCTCTGCAGCCAG
AGCTGGACTCTTTTAAGGAGGAGCTGGATAAGTACTTCAAGAATCACAC
CAGCCCCGACGTGGATCTGGGCGACATCTCTGGCATCAATGCCAGCGTG
GTGAACATCCAGAAGGAGATCGACAGACTGAACGAGGTGGCCAAGAATC
TGAACGAGAGCCTGATCGATCTGCAGGAGCTGGGCAAGTATGAGCAGTA
CATCAAGTGGCCCTGGTATATCTGGCTGGGCTTCATCGCCGGCCTGATC
GCCATCGTGATGGTGACCATCATGCTGTGCTGTATGACAAGCTGCTGTT
CCTGCCTGAAGGGCTGCTGTTCTTGTGGCAGCTGCTGTAAGTTTGATGA
GGACGATTCCGAGCCTGTGCTGAAGGGCGTGAAGCTGCACTACACCTA
A.

In order to reduce the GC-content of the gene, the inventor further optimized the codons based on NB2, resulting in a sequence designated as NB4. NB4 has a sequence as shown by SEQ ID No.: 2 below, and has 85.93% sequence identity with that of NB2.

(SEQ ID NO: 2)
ATGTTTGTCTTCCTGGTGCTGCTGCCTCTGGTCTCTTCTCAGTGTGTGA
ACCTGACCACCAGGACACAGCTGCCTCCTGCTTACACCAACTCCTTCAC
ACGGGGCGTCTACTACCCTGACAAGGTCTTCAGGTCCTCTGTGCTGCAC
TCCACACAAGACCTGTTCCTGCCATTCTTCTCCAACGTGACCTGGTTCC
ATGCCATCCATGTCTCTGGCACCAATGGCACCAAGAGGTTCGACAACCC
TGTGCTGCCATTCAACGACGGCGTCTACTTTGCCTCCACCGAGAAGTCC
AACATCATCAGGGGCTGGATCTTTGGCACCACCCTGGACTCCAAGACCC
AGTCCCTGCTGATTGTGAACAATGCCACCAACGTGGTGATCAAAGTCTG
CGAGTTCCAGTTCTGCAATGACCCATTCCTGGGCGTCTACTACCACAAG
AACAACAAGTCCTGGATGGAGTCTGAGTTCAGGGTCTACTCCTCTGCCA
ACAACTGCACCTTTGAATATGTCTCCCAGCCATTCCTGATGGACCTGGA
GGGCAAGCAGGGCAACTTCAAGAACCTGAGGGAGTTCGTCTTCAAGAAC
ATCGACGGCTACTTCAAGATCTACTCCAAGCACACACCCATTAATCTCG
TCCGGGATCTGCCTCAGGGCTTCTCTGCCCTGGAGCCCCTGGTGGACCT
GCCCATTGGCATCAACATCACACGCTTTCAGACCCTGCTGGCTCTGCAC
CGGTCTTACCTGACACCTGGCGACTCCTCCTCTGGCTGGACAGCTGGCG
CTGCTGCCTACTATGTCGGCTACCTGCAGCCTAGGACCTTCCTGCTGAA
GTACAATGAGAATGGCACCATCACAGATGCTGTGGACTGTGCCCTGGAC
CCCCTGTCTGAGACCAAGTGCACCCTGAAGTCCTTCACAGTCGAGAAGG
GCATCTACCAGACCTCCAACTTCCGGGTGCAGCCCACAGAGTCCATTGT
CAGGTTCCCCAACATCACCAACCTGTGCCCATTTGGCGAGGTCTTCAAT
GCCACACGCTTTGCCTCTGTCTATGCCTGGAACAGGAAGAGGATCTCCA
ACTGTGTGGCTGACTACTCTGTGCTGTACAACTCTGCCTCCTTCTCCAC
ATTTAAGTGCTATGGCGTCTCCCCCACCAAGCTGAATGACCTGTGCTTC
ACCAATGTCTATGCTGACTCCTTTGTGATCCGGGGCGATGAAGTTAGGC
AGATTGCCCCTGGCCAGACAGGCAAGATTGCTGACTACAACTACAAGCT
GCCTGATGACTTCACCGGCTGTGTGATTGCCTGGAACTCCAACAACCTG
GACTCTAAAGTTGGCGGCAACTACAACTACCTGTACAGGCTGTTCAGGA
AGTCCAACCTGAAGCCATTTGAGAGGGACATCTCCACAGAGATCTACCA
GGCTGGCTCCACCCCATGCAATGGCGTGGAGGGCTTCAACTGCTACTTC
CCCCTGCAATCCTATGGCTTCCAACCCACCAATGGCGTGGGCTACCAGC
CATACAGGGTGGTGGTGCTGTCCTTTGAGCTGCTGCATGCCCCTGCCAC
CGTCTGTGGCCCCAAGAAGTCCACCAACCTCGTGAAGAACAAATGTGTG
AACTTCAACTTCAACGGCCTGACAGGCACAGGCGTGCTGACAGAGTCCA
ACAAGAAGTTCCTGCCATTCCAACAGTTTGGCAGGGACATTGCTGACAC
CACCGATGCTGTCAGGGACCCCCAGACCCTGGAGATCCTGGACATCACC
CCATGCTCCTTTGGCGGCGTCTCTGTGATCACCCCTGGCACCAACACCT
CTAATCAAGTCGCTGTGCTGTACCAGGACGTGAACTGCACAGAGGTTCC
TGTGGCCATCCATGCTGACCAGCTGACCCCCACCTGGAGGGTCTACTCC
ACCGGCTCCAATGTCTTCCAAACACGGGCTGGCTGCCTGATTGGCGCTG
AGCATGTGAACAACTCCTATGAGTGTGACATCCCCATTGGCGCTGGCAT
CTGTGCCTCCTACCAAACCCAGACCAACTCCCCTCGGAGGGCTCGCTCT
GTGGCTTCTCAGTCTATCATTGCCTACACCATGTCCCTGGGCGCTGAGA
ACTCTGTGGCCTACTCCAACAACTCCATTGCCATCCCCACCAACTTCAC
CATCTCTGTGACCACCGAGATCCTGCCTGTCTCCATGACCAAGACCTCT
GTGGACTGCACCATGTACATCTGTGGCGACTCCACAGAGTGCTCCAACC
TGCTGCTGCAGTATGGCTCCTTCTGCACCCAACTGAACAGGGCCCTGAC
AGGCATTGCTGTGGAGCAGGACAAGAACACCCAGGAGGTCTTTGCCCAA
GTCAAGCAGATCTACAAGACCCCCCCCATCAAGGACTTTGGCGGCTTCA
ACTTCTCCCAAATCCTGCCTGACCCATCCAAGCCATCCAAGAGGTCCTT
CATTGAGGACCTGCTGTTCAACAAGGTTACCCTGGCTGATGCTGGCTTC
ATCAAGCAGTATGGCGACTGCCTGGGCGACATTGCTGCCAGGGACCTGA
TCTGTGCCCAAAAGTTCAATGGCCTGACAGTGCTGCCCCCCCTGCTGAC
CGATGAGATGATTGCCCAGTACACATCTGCTCTGCTGGCTGGCACAATC
ACCTCTGGCTGGACCTTTGGCGCTGGCGCTGCCCTGCAAATCCCATTTG
CCATGCAAATGGCCTACAGGTTCAATGGCATTGGCGTGACCCAGAACGT
GCTGTATGAGAACCAGAAGCTGATTGCCAACCAGTTCAACTCTGCCATT
GGCAAGATCCAGGACTCCCTGTCCTCCACCGCCTCTGCCCTGGGCAAGC
TGCAGGATGTGGTGAACCAGAATGCCCAGGCCCTGAACACCCTGGTGAA
GCAGCTGTCCTCCAACTTTGGCGCCATCTCCTCTGTGCTGAATGACATC
CTGTCCCGGCTGGACAAGGTGGAGGCTGAGGTGCAGATTGACAGGCTGA
TCACAGGCAGGCTGCAGTCCCTGCAAACCTATGTGACCCAGCAGCTGAT
CAGGGCTGCTGAGATCAGGGCCTCTGCCAACCTGGCTGCCACCAAGATG
TCTGAATGTGTGCTGGGCCAGTCCAAGAGGGTGGACTTCTGTGGCAAGG
GCTACCATCTGATGTCCTTCCCCCAATCTGCCCCCCATGGCGTGGTCTT
CCTGCATGTGACCTATGTGCCTGCTCAGGAGAAGAACTTCACCACAGCC
CCTGCCATCTGCCATGATGGCAAGGCCCACTTCCCTCGGGAGGGCGTCT
TTGTCTCCAATGGCACCCACTGGTTTGTGACCCAGAGGAACTTCTATGA
GCCTCAGATCATCACCACAGACAACACCTTTGTCTCTGGCAACTGTGAT
GTGGTGATTGGCATTGTGAACAACACAGTCTATGACCCCCTGCAGCCTG
AGCTGGACTCCTTCAAAGAGGAGCTGGACAAGTACTTCAAGAACCACAC
CTCCCCTGATGTGGACCTGGGCGACATCTCTGGCATCAATGCCTCTGTG
GTGAACATCCAGAAGGAGATTGACAGGCTGAATGAGGTGGCCAAGAACC
TGAATGAGTCCCTGATTGACCTGCAAGAGCTGGGCAAGTATGAGCAGTA
CATCAAGTGGCCATGGTACATCTGGCTGGGCTTCATTGCTGGCCTGATT
GCCATTGTGATGGTGACCATCATGCTGTGCTGCATGACCTCCTGCTGCT
CCTGCCTGAAAGGCTGCTGCTCCTGTGGCTCCTGCTGCAAGTTTGATGA
GGATGACTCTGAGCCTGTGCTGAAGGGCGTGAAGCTGCACTACACCTAA
TAA

Construction of a Vector for the Expression of the Spike Gene:

Using NB1, NB2 and NB4 as templates, respectively, the NB1 fragment was obtained through PCR amplification with primers NB1-F and NB1-R, the NB2 fragment was obtained through PCR amplification with primers NB2-F and NB2-R, and the NB4 fragment was obtained through PCR amplification with primers NB4-F and NB4-R. The vector plasmid backbone pGA1 was PCR amplified, by using CMV-R and BGH-F as primers, and using a pGA1-EGFP plasmid, which was owned by our company, as the template. Then, two-fragment in vitro recombination was carried out by using a homologous recombination enzyme (Exnase) with the NB1, NB2 or NB4 fragment, respectively, to obtain pGA1-NB1, pGA1-NB2 and pGA1-NB4. The pGA1-NB2 was linearized by using Bstz17I and SgarAI. Then, homologous recombination was carried out between the pGA1-NB2 and pAd5ΔE1ΔE3 (i.e., Ad5 empty vector), which was digested to remove the unique restriction site in the E1 region and linearized, by using competent BJ5183 cells to obtain a pAd5-NB2 vaccine vector.

The primer sequences for amplifying NB1:

NB1-F:
(SEQ ID NO.: 3)
GCGTTTAAACTTAAGCTTGGTACCGAGCTCGGATCCGCCACCATGTTT
GTTTTTCTTGT
NB1-R:
(SEQ ID NO.: 4)
AGAATAGGGCCCTCTAGACTAGTTTATGTGTAATGTAATTTG

The primer sequences for amplifying NB2:

NB2-F:
(SEQ ID NO.: 5)
GCGTTTAAACTTAAGCTTGGTACCGAGCTCGGATCCGCCACCATGTTC
GTGTTTCTGGT
NB2-R:
(SEQ ID NO.: 6)
AGAATAGGGCCCTCTAGACTAGTTTATCAGGTGTAGTGCAGCTTC

The primer sequences for amplifying NB4:

NB4-F:
(SEQ ID NO.: 7)
GCGTTTAAACTTAAGCTTGGTACCGAGCTCGGATCCGCCACCATGTTTG
TCTTCCTGGT
NB4-R:
(SEQ ID NO.: 8)
AGAATAGGGCCCTCTAGACTAGTTTAGGTGTAGTGCAGCTTC

The primer sequences for amplifying pGA1:

BGH-F:
(SEQ ID NO.: 9)
TCTAGAGGGCCCTATTCTATAGTGTC
CMV-R:
(SEQ ID NO.: 10)
GGATCCGAGCTCGGTACCAAGCTTAAGTTTAAACGCTAGAGTCCGG

PCR conditions: 95° C. for 3 min; 95° C. for 30 s; 60° C. for 30 s; 72° C. for 2 min; 30 cycles; and 72° C. for 5 min.

Rescue and Production of Ad5-NB2 Vector

1) According to a conventional method, linearizing the pAd5-NB2 vector with AsiSI, purifying by precipitation in ethanol, and transfecting into 293 cells with cationic lipofection; 8 hours after the transfection, adding 2 ml of DMEM medium containing 5% fetal bovine serum, and incubating for 7-10 days to observe cytopathic effect (CPE).

2) As observing CPE, collecting the cells and culture supernatant, repeating a freeze-thaw step in water bath at 37° C. and liquid nitrogen for 3 times, centrifuging to remove cell debris, and then infecting a 10 cm-dish with the supernatant;

3) After 2-3 days, collecting the cells and culture supernatant, repeating the freeze-thaw step for 3 times, centrifuging to remove cell debris, and then infecting 3-5 of 15 cm-dishes with the supernatant;

4) After 2-3 days, collecting the cells, repeating the freeze-thaw step for 3 times, centrifuging to remove cell debris, and then infecting 30 of 15 cm-dishes with the supernatant. After 2-3 days, collecting the cells, repeating the freeze-thaw step for 3 times, and centrifuging to remove cell debris.

5) Adding the supernatant into a cesium chloride density gradient centrifuge tube, and centrifuging at 4° C., 40000 rpm, for 4 hours;

6) Pipetting out the virus band, desalting, and subpackaging.

The virion titers were determined by absorbance at 260 nm (OD260). The calculation formula thereof was: virus concentration=OD260×dilution multiple×36/genome length (Kb). The virus stock solution was cryopreserved at −80° C.

Detection of Spike Gene Expression

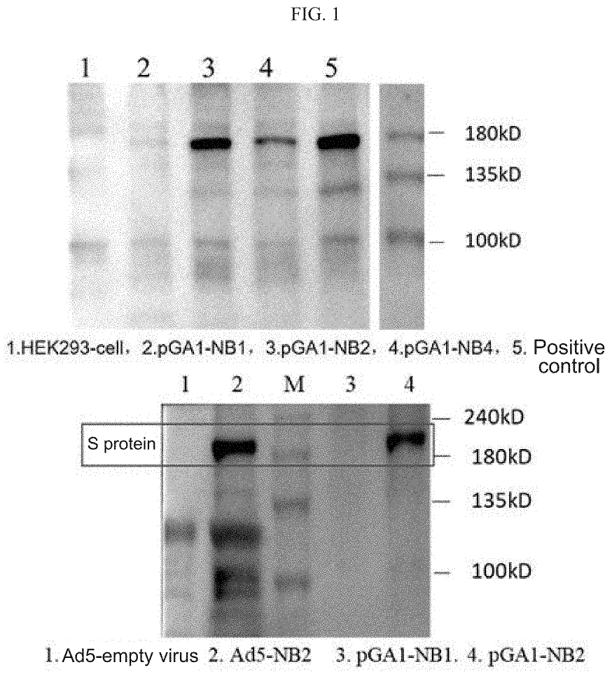

According to a conventional method, transfecting 2.5 μg of pGA-NB1, pGA-NB2 and pGA-NB4, respectively, with cationic liposomes for 48 hours, and then collecting the cells. Infecting HEK293 cells with Ad5-NB2 and the Ad5 empty vector viruses, and, after 24 hours, collecting the cells. Such four samples were processed according to a conventional Western Blot method, and were detected for the protein (FIG. 1).

It could be seen that no S protein expression was detected in the pGA1-NB1 sample, while the expression of the S protein could be observed in the codon-optimized pGA1-NB2, pGA1-NB4 and vaccine candidate strain Ad5-NB2 samples. This indicates that the NB2 sequence produces unexpected effect. The expression of the S protein could also be observed in pGA1-NB4. It indicates that a nucleotide sequence having at least 85%, further 86%, 90%, 95%, 98% and more sequence identity with NB2 has a limited expression of the corresponding protein.

Animal Immunogenicity Evaluation

Balb/c mice, 6-8 weeks old, were divided into 5 groups with 5 mice in each group. On day 0, the mice were intramuscularly immunized with Ad5-NB2 at 5×10⁹ vp/animal, through intramuscular or intra-nasal administration. On day 8, blood was taken from orbit, and serum was separated. The antibody level in the serum was detected by adopting the S protein of the novel coronavirus as the antigen through enzyme-linked immunosorbent assay (ELISA). The specific process were as follows:

1) adding 100 μl of PBS and 50 ng of the S protein per well in a 96-well plate, incubating overnight at 4° C., for 16-18 h;

2) removing the supernatant, washing with PBST once, adding 200 μl of 5% skim milk for blocking at room temperature for 2 h;

3) washing with PBST twice;

4) adding the samples: adding 100 μl of diluted serum samples, incubating at 37° C. for 2 h, and then washing with PBST for 5 times;

5) adding enzyme-labeled antibodies: adding 100 μl of diluted HRP-labeled IgM or IgG secondary antibody, and incubating at 37° C. for 2 h;

6) washing with PBST for 6-8 times;

7) adding substrate solution for development: adding 100 μl of TMB for development;

8) terminating the reaction: adding with 50 μl of 1 M sulfuric acid to terminate the reaction; and

9) determining the results: determining the OD values, and controlling the OD values between 0.1 and 4.

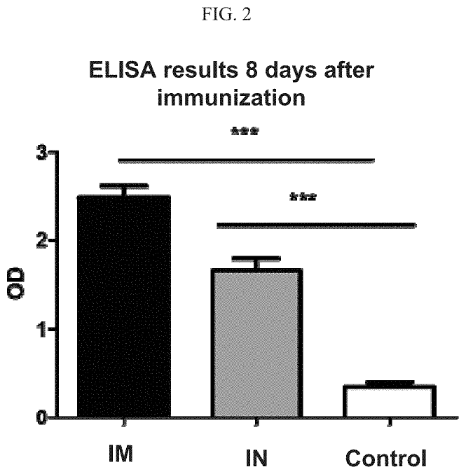

The experimental results showed that, Ad5-NB2 could induce the generation of antibodies in the mice (FIG. 2).

Immunoprotection Evaluation

The macaque monkeys were obtained from Guangdong Landau Biotechnology Co. Ltd. The vaccinated macaque monkeys were 6 to 14 years old.

The macaque monkeys were randomly divided into 4 groups, with 4 monkeys in each group, as shown in the following table:

| | | | | Time for virus |
|---|---|---|---|---|
| | | | | challenge |
| Groups | Macaca | | Immunization | (days after |
| No. | mulatta | Gender | dose/mode | immunization) |
| 1 | 063585 | Male | Ad5-NB2 (1 × 10¹⁰ vp) | 30 |
| | 071515 | Male | Intramuscular (IM) | |
| | 080066 | Female | injection | |
| | 134056 | Female | | |
| 2 | 100109 | Male | Ad5-NB2 (1 × 10¹¹ vp) | 56 |
| | 116004 | Male | Intramuscular (IM) | |
| | 116008 | Female | injection | |
| | 130460 | Female | | |
| 3 | 071539 | Male | Ad5-NB2 (1 × 10¹¹ vp), | 30 |
| | 110113 | Male | intra-nasal/oral (I.N.) | |
| | 140052 | Female | | |
| | 134018 | Female | | |
| 4 | 100886 | Female | Ad5-empty | 30 |
| | 110075 | Male | (1 × 10¹¹ vp) | |
| 5 | C1 | Female | — | — |
| | C2 | Male | | |
| | C3 | Female | | |
| | C4 | Male | | |
| | D1 | Female | | |
| | D2 | Male | | |

Note:
the intra-nasal/oral immunizing dose for each macaque monkey was 5 × 10¹⁰ vp.

Macaque monkeys were immunized with the vaccine prepared from the Ad5-NB2 virus strain. Blood was taken at days 12, 18 and 24 after the immunization, to determine the antibody binding titers by ELISA, and the neutralizing antibody titers by the SARS-CoV-2 virus. The peripheral blood was isolated and detected for cellular immune reaction by ELISpot assay.

### EXPERIMENTAL RESULTS

(1) Binding Antibodies

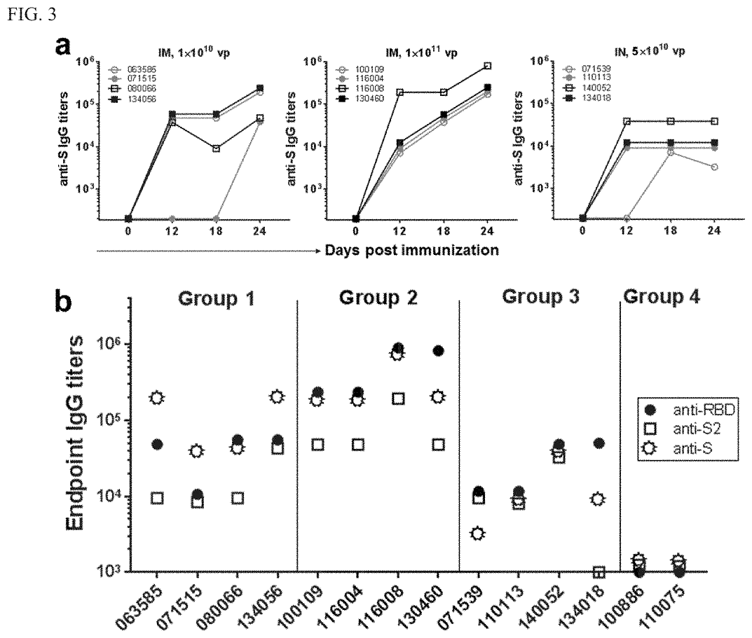

12 days after the intramuscular (I.M.) vaccination, significant presence of S-specific IgG was detected in the sera of 4 macaque monkeys injected with 1×10¹¹ vp and 3 macaque monkeys injected with 1×10¹⁰ vp. After 24 days, S-specific IgG was detected in the sera of all immunized macaque monkeys. In the macaque monkeys immunized with a low or high dose vaccine, the S-specific IgG increased continuously over time (FIG. 3a).

12 days after the intra-nasal/oral vaccination, the S-specific IgG was only detected in the sera of 3 out of 4 vaccinated macaque monkeys. On 24th day, the 5-specific IgG was detected in the sera of all immunized macaque monkeys.

Compared with intramuscular vaccination, the serum IgG titer reached the peak on 18th day after intra-nasal/oral vaccination, 1-2 logarithms lower than that of the macaque monkeys intramuscularly immunized with the same dose of the vaccine (FIGS. 3a and 3b).

(2) Neutralizing Antibodies

The neutralizing antibody titers of the serum samples were determined by means of the neutralization reaction between a live novel coronavirus (2019-nCoV-WIV04 strain) and serum antibodies.

Heat-inactivating the obtained animal serum samples at 56° C. for 30 min; diluting by 1:50, 1:150, 1:450, 1:1350, 1:4050 and 1:12150, respectively; adding the equal amount of live viruses and incubating at 37° C. under 5% CO2 for 3 days; and then fixing the cells with 4% formaldehyde and staining with crystal violet. The number of plaques was counted to determine the neutralizing antibody titer (EC₅₀).

The results showed (FIG. 4) that: 28 days after the immunization, the sera of the macaque monkeys injected intramuscularly with 1×10¹¹ vp had neutralizing antibodies against live novel coronaviruses; and the sera of the macaque monkeys with intra-nasal/oral immunization (5×10¹⁰ vp/animal) also had neutralizing antibodies against live novel coronaviruses, which had lower titer than that of the intramuscularly immunized macaque monkeys. 56 days after the immunization, the sera of the macaque monkeys injected intramuscularly with 1×10¹⁰ vp also had neutralizing antibodies against live novel coronaviruses, which had lower titer than that of the macaque monkeys injected intramuscularly with 1×10¹⁰ vp.

(3) Cellular Immunity

To determine whether Ad5-NB2 could also induce a cellular immune response in non-human primates (NHPs), we detected the response of S-specific IFN-γ secreting cells from the peripheral blood mononuclear cells (PBMCs), to 51 and S2 peptide libraries.

The results showed that:

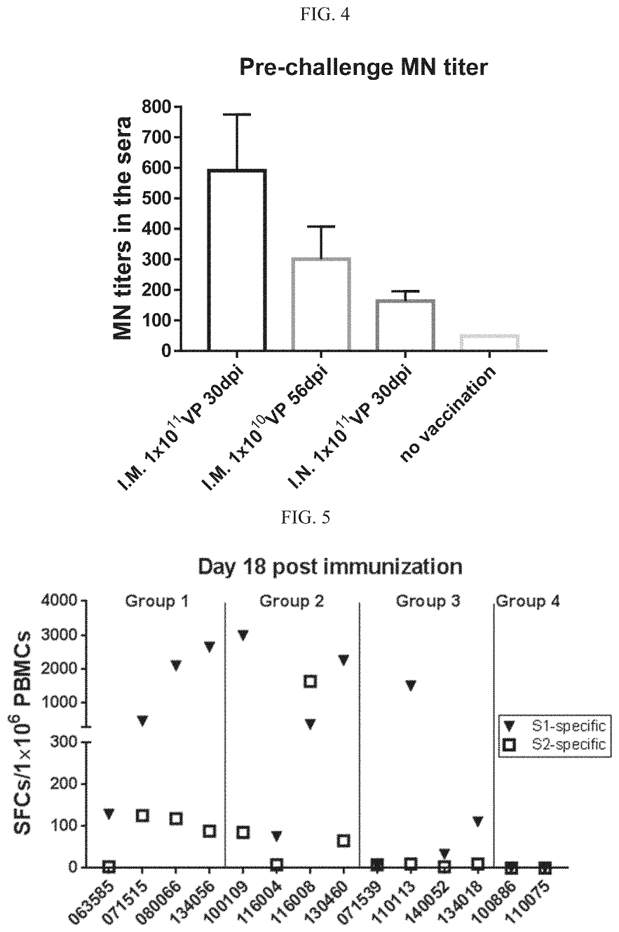

1) On the 18th day after the intramuscular vaccination, all 8 macaque monkeys had cellular immune responses to the 51 peptide library, and 6 macaque monkeys had cellular immune responses to the S2 peptide library (FIG. 5).

2) On the 18th day after the intra-nasal/oral vaccination, 2 out of 4 vaccinated macaque monkeys showed weak cellular immune responses to the S1 peptide library, and all 4 vaccinated macaque monkeys had no obvious response to the S2 peptide library on the 18th day (FIG. 5).

Therefore, in the macaque monkeys, the cellular immune response was mainly directed at the S1 region, which was similar to that observed in mice. These results indicates that the intramuscular immunization of the vaccine can cause systemic cellular immune response to the S protein, especially to the S1 region. While the mucosal vaccination of the vaccine causes weaker systemic cellular immune response.

Protective Efficacy Test (Virus Challenge Tests in Macaque Monkeys)

The virus challenge tests were carried out in the P4 Laboratory of Wuhan Institute of Virology, Chinese Academy of Sciences. At 30 days for the group immunized with 1×10¹¹ VP (IM. and I.N. vaccination groups), and 8 weeks for the group immunized with a low dose of 1×10¹⁰ VP (IM. vaccination group) after the vaccination, the animals were challenged with 2×10⁴ TCID50 SARS-CoV-2 (2019-nCoV-WIV04) by intratracheal route. Due to the limited space of the P4 Laboratory, we only randomly selected 3 macaque monkeys of the I.M. high-dose vaccinated group, 3 macaque monkeys of the I.N. high-dose vaccinated group, and 3 macaque monkeys of the I.M. low-dose vaccinated group, for the virus challenge test. At the same time, 4 non-vaccinated macaque monkeys (C1-C4) received the virus challenge with the same dose of viruses, and the other 2 non-vaccinated macaque monkeys (D1 and D2) received the virus challenge at the dose of 400 TCID50. The nucleic acids from ten sites, such as pharyngeal swab, bronchus and lung lobe, were collected and detected for 10 consecutive days after the virus challenge. The detection results showed that:

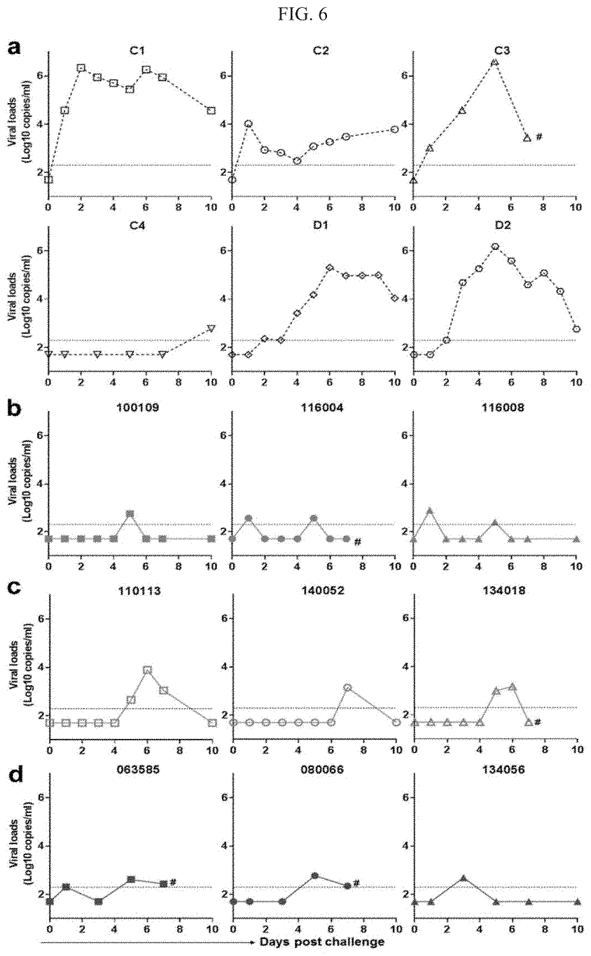

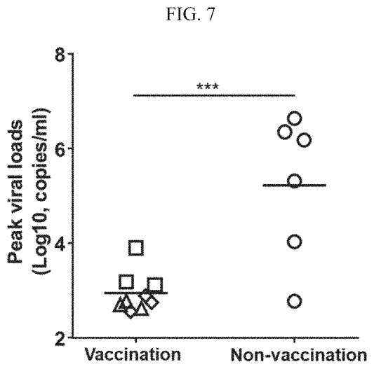

1) With genome detection of the novel coronaviruses, no virus was detected at the pharyngeal swabs of the immunized macaque monkeys after virus challenge, or a very low number of copies of the virus was detected at some sites.
For the non-vaccinated macaque monkeys (C1-C4, D1-D2), a high load of novel coronavirus nucleic acids was detected in the pharyngeal swabs, and the viral load in the pharyngeal swabs climbed to a peak of 4.2×10⁶ copies/ml on the 5th day after the virus challenge (FIG. 6 a).
For the intramuscular vaccination group of a high dose of 1×10¹¹ vp, the nucleic acids on the pharyngeal swabs were detected by qPCR quantitative tests 30 days after immunization. The results showed that, the viral loads of some of the pharyngeal swabs of the macaque monkeys were slightly higher than the detection limit only on day 1 or day 5, and the other viral loads were undetectable. Therefore, the SARS-CoV-2 infection can be effectively prevented by intramuscular vaccination of 1×10¹¹ vp of Ad5-NB2 (FIG. 6b).
For the intramuscular vaccination group of a low dose of 1×10¹⁰ vp, the nucleic acids on the pharyngeal swabs were detected by qPCR quantitative tests 56 days (8 weeks) after immunization. The result showed that the viral loads of some of the pharyngeal swabs of the macaque monkeys were slightly higher than the detection limit only on day 3 or day 5, and the other viral loads were undetectable. Therefore, the SARS-CoV-2 infection can be effectively prevented by intramuscular vaccination of 1×10¹⁰ vp of Ad5-NB2 (FIG. 6c).
For the intra-nasal/orally immunized macaque monkeys, the virus was undetectable in all of macaque monkeys in the first 4 days after the virus challenge, and the viral loads from some of the macaque monkeys were slightly higher than the detection limit on day 6 or day 7 and then decreased, and were undetectable on day 10. Therefore, the intra-nasal/oral immunization can also effectively resistant to the SARS-CoV-2 infection (FIG. 6d).

In view of the above, all immunized macaque monkeys were fully protected, and effectively resistant to the SARS-CoV-2 infection. The systemic antibodies and cellular immune responses induced by the intramuscular vaccination in the macaque monkeys was significantly higher than those induced by the mucosal vaccination.

2) No sign was found for the antibody-dependent enhancement (ADE) in all vaccinated macaque monkeys after virus challenge.

We calculated the viral loads based on the area under curve (AUC). The AUCs (2.7±0.6 log₁₀) of the viruses of the vaccinated macaque monkeys was 2500 times lower than those (6.1±1.0 log₁₀) of the non-vaccinated macaque monkeys (see FIG. 7).

3) After euthanasia, the virus genomes in the biopsy samples from nine anatomic sites (including trachea, left and right bronchi, upper, middle and lower parts of left and right lungs) of the macaque monkeys were evaluated.

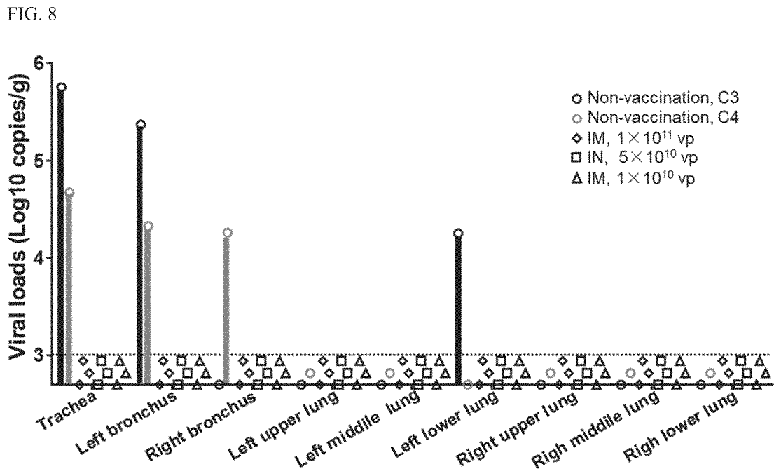

The results showed that: the virus genomes were detected in the left bronchus (2.4×10⁵ copies/ml) and the lower part of left lung (1.8×10⁴ copies/ml) of the non-vaccinated macaque monkey C3; the virus genomes were also detected in the trachea (4.7×10⁴ copies/ml), left (2.2×10⁴ copies/ml) and right bronchi (1.8×10⁴ copies/ml) of the non-vaccinated macaque monkey C4. In contrast, it showed that the results were all negative through the novel coronavirus nucleic acid test performing on 81 biopsy samples collected from 9 vaccinated macaque monkeys (FIG. 8).

4) Pathological sections confirmed that the lungs of the immunized macaque monkeys were effectively protected.

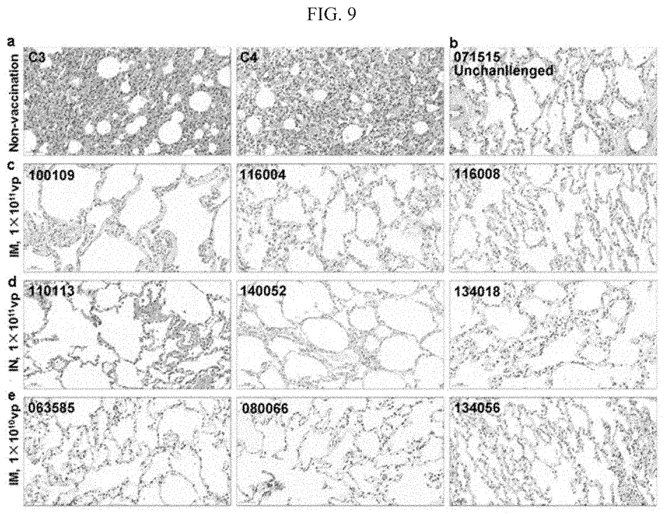

Histopathological analysis was carried out on the lung slices of the non-vaccinated and vaccinated macaque monkeys. In the non-SARS-CoV-2-vaccinated macaque monkeys, SARS-CoV-2 intratracheal inoculation caused severe interstitial pneumonia, which was manifested by alveolar septum dilatation, infiltration of monocytes and lymphocytes in most alveoli, and a certain proportion of alveolar edema. In the vaccinated macaque monkeys, no obvious pathological abnormality was caused by the virus challenge test. Some of the macaque monkeys had slight histopathological changes in the lungs, which might be due to mechanical damages caused by direct endotracheal intubation for performing virus challenge to the macaque monkeys (FIG. 9).

5) Titer changes of serum antibody of the macaque monkeys before and after immunization

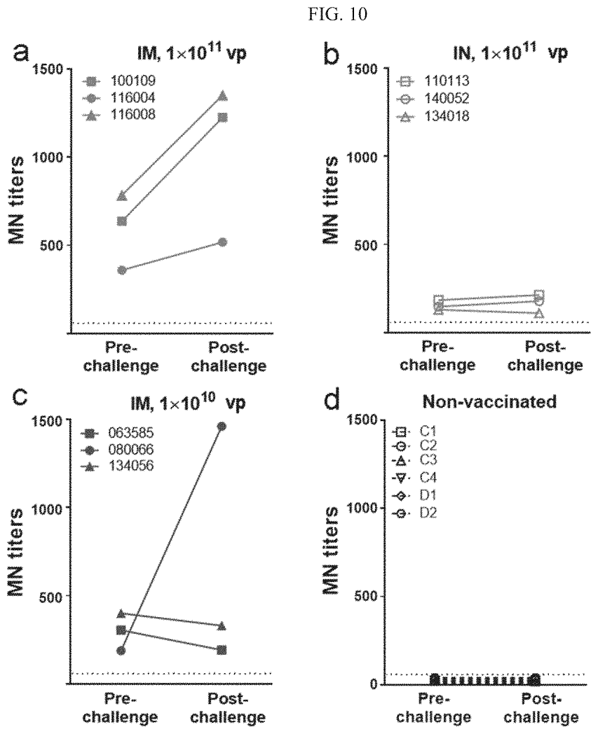

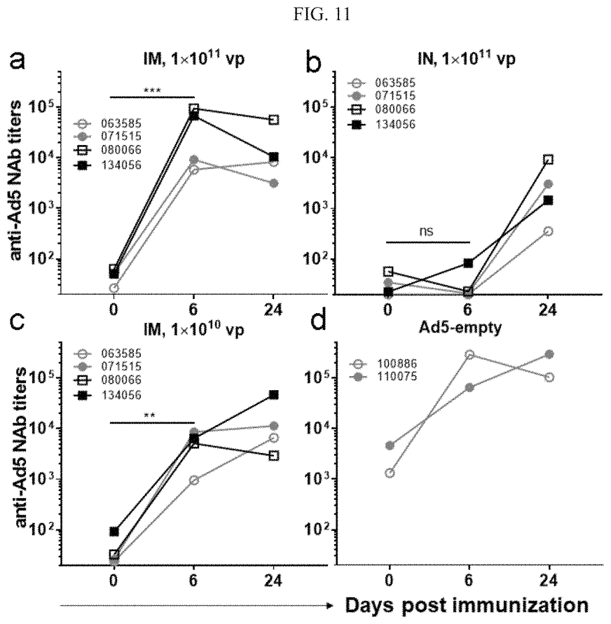

A plaque reduction neutralization test (PRNT) was performed for comparing the titers of neutralizing antibodies in the sera before and after the virus challenge.
All non-vaccinated macaque monkeys did not show SARS-CoV-2-neutralizing activity in sera before virus challenge or at 7 days after virus challenge (<1:50) (FIG. 10d).
For the intramuscular vaccination group of a high dose, the titers of neutralizing antibodies in the sera were respectively 1:636, 1:389 and 1:784 before virus challenge, and were respectively 1:1225, 1:518 and 1:1350 on day 7 after virus challenge. It indicates that the virus challenge has no or low enhancement effect (FIG. 10a).
For the intramuscular vaccination group of a low dose, only one macaque monkey (No. 080066) had significantly increased titer of neutralizing antibody in the serum (IC50 1:188→1:1460) on day 7 after virus challenge, while the other two macaque monkeys had no increase in the titer of neutralizing antibodies in the sera (IC50 1:315→1:192, 1: 400→1:330), as compared with those before the virus challenge (FIG. 10c).
For the intra-nasal/oral vaccination group, the titers of neutralizing antibodies in the sera were relatively low both before the virus challenge and on day 7 after the virus challenge, without significant change. The titers of neutralizing antibodies in the sera were respectively 1:164, 1:137 and 1:150 before virus challenge, and were respectively 1:193, 1:168 and 1:130 on day 7 after virus challenge (FIG. 10b).
The levels of anti-Ad5 neutralizing antibodies were detected in the Ad5-NB2-immunized macaque monkeys. The results showed that the titers of Ad5-neutralizing antibodies in the sera were increased on day 6 after Ad5-NB2 immunization in the intramuscular vaccination group. However, for the intra-nasal/orally Ad5-NB2-immunized group, the titers of Ad5-neutralizing antibodies in the sera were not increased significantly on day 6 after the immunization, and were increased on day 24, but not higher than those in the group of a low dose (FIG. 11a-11d).

In view of the above, in 6 out of 9 macaque monkeys from the immunized groups, the titers of neutralizing antibodies against the novel coronavirus did not increase significantly before and after the virus challenge. It suggested that the virus was eliminated immediately and no obvious virus replication occurred.

In summary, the optimized S gene, NB2, in the replication-defective type 5 adenovirus vector can significantly improve antigen expression, and induce high-titer antibodies and cellular immunity after immunizing mice and macaque monkeys (Macaca mulatta). The macaque monkeys were immunized by single intramuscular or intra-nasal/oral administration of the vaccine, and were fully resistant to the virus infection after being subjected to virus challenge 30 days after the immunization. When the macaque monkeys were intramuscularly immunized once with 1/10 low dose (1×10¹⁰ vp/animal) of the vaccine and then subjected to virus challenge 8 weeks after the immunization, the macaque monkeys were also fully resistant to the virus infection. When the vaccinated macaque monkeys were subjected to virus challenge, we did not observe lung injury caused by ADE or virus. In most of the vaccinated macaque monkeys, the antibody level did not increase after virus challenge, which indicated that the virus was eliminated quickly and did not cause stimulation effect on the immune system. Different from the whole-virus inactivated vaccine, the vaccine of the present disclosure, in addition to stimulating the humoral immunity of a body, also induces cellular immunity, which can further improve the protective effect on body function. Furthermore, since the nasal and oral immunization also obtained good protective effect, it suggests the feasibility of mucosal- and non-injection immunization.

1. A vaccine for preventing Novel Severe Acute Respiratory Syndrome Coronavirus 2 (SARS-CoV-2) infection, comprising:
a nucleotide sequence as shown by SEQ ID NO: 1; or
a nucleotide sequence having at least 80%, 85%, 90%, 95% or 100% homology with the nucleotide sequence as shown by SEQ ID NO: 1.

2. The vaccine according to claim 1, wherein the nucleotide sequence is capable of expressing a protein in a human-derived cell or human body.

3. The vaccine according to claim 2, wherein, in the human body, the protein is capable of:
inducing an immune response;
generating a molecular biology reporter;
generating a trace molecule for detection;
regulating a gene function; or
functioning as a therapeutic molecule.

4. The vaccine according to claim 1, further comprising a pharmaceutically-acceptable adjuvant, carrier, diluent or excipient.

5. The vaccine according to claim 1, further comprising at least one medicament having a therapeutic effect on coronavirus disease 2019 (COVID-19).

6. An expression vector comprising the nucleotide sequence according to claim 1.

7. The expression vector according to claim 6, wherein the vector is a DNA plasmid, an RNA expression plasmid or a viral vector.

8. The expression vector according to claim 7, wherein the viral vector is an adenovirus vector.

9. The expression vector according to claim 8, wherein the nucleotide sequence according to claim 1 has a transcription direction opposite to those of other genes in the adenovirus vector.

10. The vaccine according to claim 1, wherein the vaccine is capable of:
preparing a COVID-19 detection reagent; or
preparing a gene-function regulator.

11. A method for preventing or treating COVID-19, comprising administering a prophylactically or therapeutically effective amount of the vaccine according to claim 1 to a human.

13. The method according to claim 12, wherein in the human body, the protein is capable of:
inducing an immune response;
generating a molecular biology reporter;
generating a trace molecule for detection;
regulating a gene function; or
functioning as a therapeutic molecule.

14. The method according to claim 11, wherein the vaccine further comprises a pharmaceutically-acceptable adjuvant carrier, diluent or excipient.

15. The method according to claim 11, wherein the vaccine further comprises at least one medicament having a therapeutic effect on coronavirus disease 2019 (COVID-19).

16. The vaccine according to claim 2, further comprising a pharmaceutically-acceptable adjuvant, carrier, diluent or excipient.

17. The vaccine according to claim 3, further comprising a pharmaceutically-acceptable adjuvant, carrier, diluent or excipient.

18. The vaccine according to claim 2, further comprising at least one medicament having a therapeutic effect on coronavirus disease 2019 (COVID-19).

19. The vaccine according to claim 3, further comprising at least one medicament having a therapeutic effect on coronavirus disease 2019 (COVID-19).
